(19) United States
(12) Patent Application Publication
(10) Pub. No.: US 2023/0411022 A1
(43) Pub. Date: Dec. 21, 2023

(54) INTELLIGENT QUESTION-ANSWERING SYSTEM FOR PROSTATE CANCER AND IMPLEMENTATION METHOD THEREOF

(71) Applicant: West China Hospital, Sichuan University, Chengdu (CN)

(72) Inventors: Bairong Shen, Chengdu (CN); Tong Tang, Chengdu (CN); Jiao Wang, Chengdu (CN); Xingyun Liu, Chengdu (CN); Rongrong Wu, Chengdu (CN); Mengqiao He, Chengdu (CN); Fei Ye, Chengdu (CN); Yingbo Zhang, Chengdu (CN)

(21) Appl. No.: 18/315,412

(22) Filed: May 10, 2023

(30) Foreign Application Priority Data
May 19, 2022 (CN) 2022105456443

(51) Int. Cl. G16H 70/60; G16H 10/60; G16H 15/00

(52) U.S. Cl. CPC G16H 70/60; G16H 15/00; G16H 10/60

(57) ABSTRACT

An implementation method of an intelligent question-answering system for prostate cancer includes: acquiring lifestyle data for prostate cancer based on a preset first data source; using the lifestyle data for prostate cancer as metadata to construct a lifestyle knowledge base; constructing a lifestyle knowledge graph based on a preset second data source and the lifestyle knowledge base; and fusing the lifestyle knowledge base and the lifestyle knowledge graph to obtain the intelligent question-answering system for prostate cancer. The intelligent question-answering system for prostate cancer can help clinicians, medical staff, scientific researchers, and patients, as well as the general public, to acquire objective lifestyle data conveniently and efficiently, and accurately assess the impact on prostate cancer.

### TECHNICAL FIELD

The disclosure relates to the field of biomedicine and computer storage technology, and more particularly to an intelligent question-answering system for prostate cancer and its implementation method.

### BACKGROUND

According to global cancer burden data published by the International Agency for Research on Cancer (IARC) of the World Health Organization, prostate cancer is one of the most common tumors in men, it not only brings great physical and mental pain to patients, such as erectile dysfunction, difficulty urinating, hematuria, fatigue, etc., but also brings huge economic pressure and burden to the patient families. In China, the prevalence rate of prostate cancer is increasing year by year with the increasing local environmental pollution, the increasing pressure of urban life, irregular work and rest time, and other factors. Relevant academic research organizations discovered that lifestyle is an important way to affect incidence and treatment of prostate cancer. At present, there already exists a Lifestyle Database for Precision Prevention of Prostate Cancer (PCaListDB). A total of 3024 lifestyles related to prostate cancer were collected in the PCaListDB, it includes 394 protective lifestyles, 556 risky lifestyles, 45 non-influencing lifestyles, 52 confounding lifestyles, and 1977 lifestyles lacking sufficient data support.

However, since this traditional knowledge base mainly provides scientific data to users in the form of tables, and cannot provide basic information on lifestyle and clinical information such as prostate cancer pathological staging, it greatly limits its use in non-scientific research users, such as patients and the general public.

### SUMMARY

The technical problem to be solved by the disclosure is to provide an intelligent question-answering system for prostate cancer and its implementation method, it can help clinicians, medical staff, scientific researchers, and patients, as well as the general public, to acquire objective lifestyle data conveniently and efficiently, and accurately assess the impact on prostate cancer.

In order to solve the above technical problem, on the one hand, the disclosure discloses an implementation method of an intelligent question-answering system for prostate cancer, the method includes: acquiring lifestyle data for prostate cancer based on a preset first data source; constructing a lifestyle knowledge base by taking the lifestyle data for prostate cancer as metadata; constructing a lifestyle knowledge graph based on a preset second data source and the lifestyle knowledge base; and fusing the lifestyle knowledge base and the lifestyle knowledge graph to obtain the intelligent question-answering system for prostate cancer.

In an embodiment, acquiring the lifestyle data for prostate cancer based on the preset first data source, includes: training a keyword model related to the lifestyle data for prostate cancer; modularizing data of the first data source to obtain a plurality of datasets; and extracting the lifestyle data for prostate cancer from the plurality of datasets by using the keyword model.

In an embodiment, constructing the lifestyle knowledge base by taking the lifestyle data for prostate cancer as the metadata, includes: standardizing the lifestyle data for prostate cancer to obtain standard data; and structuring the standard data to obtain the lifestyle knowledge base with index relationships.

In an embodiment, constructing the lifestyle knowledge graph based on the preset second data source and the lifestyle knowledge base, includes: filtering data of the second data source to obtain prostate science popularization data related to the lifestyle data for prostate cancer by using the keyword model; and allocating the prostate science popularization data to obtain the lifestyle knowledge graph according to a preset graph rule.

In an embodiment, fusing the lifestyle knowledge base and the lifestyle knowledge graph to obtain the intelligent question-answering system for prostate cancer, includes: taking the prostate science popularization data in the lifestyle knowledge graph and the lifestyle data for prostate cancer in the lifestyle knowledge base as a corpus dataset, the corpus dataset at least including prostate cancer question texts and prostate cancer answer texts; performing semantic parsing on each of the prostate cancer question texts in the corpus dataset to obtain a user intention of each prostate cancer question text, determining a coverage of the prostate cancer question texts in the corpus dataset based on the user intention of each prostate cancer question text, and classifying each prostate cancer question text in the corpus dataset based on the user intention of each prostate cancer question text to obtain a category attribute corresponding to each prostate cancer question text; and constructing a question-answering knowledge base based on the coverage of the prostate cancer question texts in the corpus dataset and the corresponding category attribute of each prostate cancer question text.

According to the second aspect of the disclosure, an intelligent question-answering system for prostate cancer is provided, and the system includes: an acquisition module, a lifestyle knowledge base and a lifestyle knowledge graph. The acquisition module is configured to acquire lifestyle data for prostate cancer based on a preset first data source; the lifestyle knowledge base is constructed by taking the lifestyle data for prostate cancer as metadata; the lifestyle knowledge graph is constructed based on a preset second data source and the lifestyle knowledge base; and the intelligent question-answering system for prostate cancer is implemented by fusing the lifestyle knowledge base and the lifestyle knowledge graph.

In an embodiment, each of the acquisition module, the lifestyle knowledge base and the lifestyle knowledge graph is embodied by software stored in at least one memory and executable by at least one processor.

In an embodiment, the acquisition module includes a keyword model trained to be related to the lifestyle data for prostate cancer; and the acquisition module is configured to modularize data of the first data source to obtain a plurality of datasets, and extract the lifestyle data for prostate cancer from the plurality of datasets by using the keyword model.

In an embodiment, the lifestyle knowledge base is implemented by standardizing the lifestyle data for prostate cancer to obtain standard data and structuring the standard data to obtain the lifestyle knowledge base with index relationships.

In an embodiment, the lifestyle knowledge graph is implemented by filtering data of the second data source to obtain prostate science popularization data related to lifestyle data for prostate cancer by using the keyword model and allocating the prostate science popularization data to obtain the lifestyle knowledge graph according to a preset graph rule.

In an embodiment, the intelligent question-answering system for prostate cancer is implemented by fusing the lifestyle knowledge base and the lifestyle knowledge graph, specifically includes: taking the prostate science popularization data in the lifestyle knowledge graph and the lifestyle data for prostate cancer in the lifestyle knowledge base as a corpus dataset, the corpus dataset at least including prostate cancer question texts and prostate cancer answer texts; performing semantic parsing on each of the prostate cancer question texts in the corpus dataset to obtain a user intention of each prostate cancer question text, determining a coverage of the prostate cancer question texts in the corpus dataset based on the user intention of each prostate cancer question text, and classifying each prostate cancer question text in the corpus dataset based on the user intention of each prostate cancer question text to obtain a category attribute corresponding to each prostate cancer question text; and constructing the question-answering knowledge base based on the coverage of the prostate cancer question texts in the corpus dataset and the corresponding category attribute of each prostate cancer question text.

Compared with the related art, the beneficial effects of the disclosure are as follows.

The disclosure can respectively construct the lifestyle knowledge base and the lifestyle knowledge graph through the introduction of dual data sources, greatly expanding the scientific capacity and data sources of the database and improving the stability and objectivity of the data sources, furthermore, the lifestyle knowledge base and the lifestyle knowledge graph are limited to the lifestyle data related to prostate cancer, it's able to more objectively approach the data situation that the data demand side wants to understand, furthermore the intelligent question-answering system fused by these two ways can enable users to use it autonomously, it is not only convenient for clinicians, scientific researchers and other professionals, but more importantly, it is convenient for patients, residents, the elderly and other general public to scientifically and effectively understand and inquire about the occurrence, development, treatment and prognosis of prostate cancer, providing an opportunity.

### BRIEF DESCRIPTION OF DRAWINGS

FIG. 1 is a flowchart of an implementation method of an intelligent question-answering system for prostate cancer according to an embodiment of the disclosure.

FIG. 2 is a schematic diagram of an application of an intelligent question-answering system for prostate cancer according to an embodiment of the disclosure.

FIG. 3 is a block diagram of an intelligent question-answering system for prostate cancer according to an embodiment of the disclosure.

FIG. 4 is a schematic structural diagram of an intelligent question-answering device for prostate cancer according to an embodiment of the disclosure.

### DETAILED DESCRIPTION OF EMBODIMENTS

In order to better understand and implement, the technical solutions in embodiments of the disclosure will be clearly and completely described in conjunction with the accompanying drawings. The described embodiments are only some of the embodiments of the disclosure, not all of them. Based on the embodiments in the disclosure, all other embodiments acquired by those skilled in the art without creative work fall within the scope of protection of the disclosure.

The terms “including” and “having” and any variations thereof in the embodiments of the disclosure are intended to cover non-exclusive inclusion, for example, a process, method, system, product, or device that includes a series of steps or modules needs not be limited to those clearly listed steps or modules, but may include other steps or modules that are not clearly listed or inherent to the process, method, product, or device.

The embodiments of the disclosure disclose an intelligent question-answering system for prostate cancer and an implementation method of intelligent question-answering system, the disclosure can construct the lifestyle knowledge base and the lifestyle knowledge graph through the introduction of dual data source, greatly expanding the scientific capacity and data sources of database and improving the stability and objectivity of the data sources, furthermore the lifestyle knowledge base and the lifestyle knowledge graph are limited to the lifestyle data related to prostate cancer, it's able to more objectively approach the data situation that the data demand side wants to understand, furthermore the intelligent question-answering system fused by these two ways can enable users to use it autonomously, it is not only convenient for clinicians, scientific researchers and other professionals, but more importantly, it is convenient for patients, residents, the elderly and other general public to scientifically and effectively understand and inquire about the occurrence, development, treatment and prognosis of prostate cancer, providing an opportunity.

### Embodiment 1

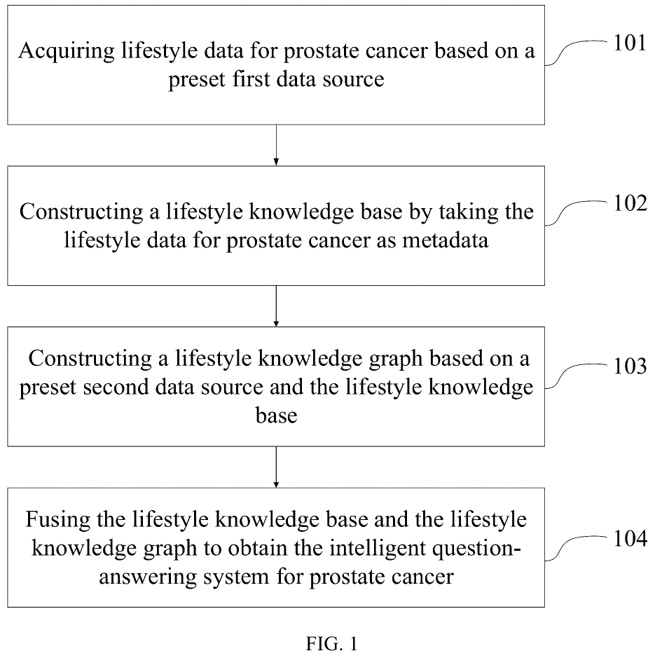

Please refer to FIG. 1, FIG. 1 is a flowchart of an implementation method of an intelligent question-answering system for prostate cancer according to an embodiment of the disclosure. Specifically, the implementation method of the intelligent question-answering system for prostate cancer can be applied to chat robot terminals, mobile device terminals, portable mobile device terminals, personal wearable device terminals, web page terminals, etc. The application of this implementation method is not limited by the embodiments of the disclosure. As shown in FIG. 1, the implementation method of the intelligent question-answering system for prostate cancer can include the following steps.

101. Acquiring lifestyle data for prostate cancer based on a preset first data source.

First of all, in order to build a base structure for the intelligent question-answering system for prostate cancer, the preset first data source is used as a basic data source. In an embodiment, the preset first data source is implemented by objective global academic literature systems and websites, exemplarily, in the embodiment, the first data source can refer to the National Center for Biotechnology Information (NCBI) literature database and the Chinese National Knowledge Infrastructure (CNKI) literature database. After that, data from the above databases are invoked through a data source interface, due to the inventor found that the impact of lifestyle on prostate cancer is of utmost importance, in these databases, the lifestyle data for prostate cancer are mainly filtered, and the lifestyle data for prostate cancer can refer to the lifestyle data affected the probability and clinical outcomes of prostate cancer, the lifestyle data not only include names, types, subclasses, doses and exposure time of the lifestyle and disease classification and impact relationship of prostate cancer, but also include background information such as the population, race, quantity, data sources, literature names, publication dates, years, and journals involved in the research. Exemplarily, the lifestyle data include personal background characteristics (i.e., family history, etc.), behavioral habits (i.e., smoking, alcohol, etc.), environmental factors (i.e., bisphenol A, insecticides, etc.), minerals (i.e., selenium, calcium, etc.), vitamins (i.e., serum retinol (β-Carotenoids, etc.), drugs (i.e., antidiabetic, statins, etc.), diseases (i.e., diabetes, metabolic syndrome, etc.), social factors (i.e., long-term work, stress, etc.), food (i.e., carbohydrate, fat intake, etc.), physiological and biochemical factors (i.e., endocrine, hormone), etc. The data are manually retrieved, collected and extracted, cleaned, filtered, and structured, the content of data collection not only include names, types, subclasses, doses and exposure times of lifestyle, as well as disease classification and impact relationships of prostate cancer, but also include background information such as the population, race, quantity, data sources, literature names, publication dates, years, and journals involved in the research.

The acquiring lifestyle data for prostate cancer is implemented by: training a keyword model related to the lifestyle data for prostate cancer based on a preset machine learning training method, the keyword model includes multiple keywords and keyword-related statements of the above lifestyle data. The training model (i.e., keyword model) can be continuously updated and upgraded based on the update from the first data source, and then, the data from a calling interface of the first data source is modularized to obtain multiple datasets. Modularization is the most important feature of metadata, the key of the modularization is to divide resource objects into several entities according to the actual use needs. The description of resource refers to combination and description of multiple different entities, that is multiple datasets of different source types can be obtained, and the lifestyle data for prostate cancer can be extracted from the multiple datasets by using the trained keyword model. The extracted method can include inputting the datasets into the training model, performing automatically outputting on the matched lifestyle data for prostate cancer based on history training results.

102. Constructing a lifestyle knowledge base by taking the lifestyle data for prostate cancer as metadata.

In order to achieve user end conversion of the acquired lifestyle data for prostate cancer, the lifestyle data for prostate cancer is standardized to obtain standard data, the standardized method can be implemented by manually searching, collecting and extracting, cleaning, and filtering to uniformly define identifiers, data types, data lengths, and other information for different sources of data. After that, the standard data is structured to obtain the lifestyle knowledge base with index relationships, the structuration is primarily aimed at the user, the standard data can be transformed into recognizable and understandable data for the system, exemplarily, standardized texts of the metadata is transformed to XML Schema formal description files, various resource metadata is transformed and encapsulated to XML files based on XML Schema, so as to support automatic identification, understanding and verification of the XML files by computers. On the basis of the above data preparation, scientific relationships between various lifestyles and prostate cancer are sorted out manually to form an index relationship list, so as to form the lifestyle knowledge base.

103. Constructing a lifestyle knowledge graph based on a preset second data source and the lifestyle knowledge base.

In an embodiment, in order to strengthen the objectivity of data, the embodiment uses the second data source as an access method of data source, further concretizing the lifestyle knowledge base. The second data source can be derived from authoritative and widely global science popularization platforms such as Wikipedia. The data are acquired through the second data source to increase the data diversity and authority of the lifestyle knowledge base. Specifically, the data can include basic information about each lifestyle acquired from the second data source, the basic information includes synonyms, sources, basic descriptions, and other information about lifestyle, the data can further include the description, staging, Gleason score, and other information of prostate cancer in the clinical guidelines of prostate cancer. The acquired method can include filtering data of the second data source to obtain prostate science popularization data related to the lifestyle data for prostate cancer by using the keyword model, and allocating the prostate science popularization data to obtain the lifestyle knowledge graph according to a preset graph rule, the graph rule can include an inference rule, the graph rule can include using description logic for description. The description logic is a formal knowledge expression method based on logic. The description logic defines concepts, relationships, entities and a series of operators used to describe and constrain entity relationships, so as to establish a relationship between the acquired data and prostate cancer, and construct the lifestyle knowledge graph by using the relationship.

104. Fusing the lifestyle knowledge base and the lifestyle knowledge graph to obtain the intelligent question-answering system for prostate cancer.

After acquiring two objective dimensions of the lifestyle knowledge base and the lifestyle knowledge graph, it is necessary to transform these two types of data into the intelligent question-answering system, and the system can interact with users, specifically, the prostate science popularization data in the lifestyle knowledge graph and the lifestyle data for prostate cancer in the lifestyle knowledge base are taken as a corpus dataset, and the corpus dataset at least includes prostate cancer question texts and prostate cancer answer texts. Then semantic parsing is performed on each of the prostate cancer question texts in the corpus dataset to obtain a user intention of each prostate cancer question text, the semantic parsing can be implemented through alphabetic string parsing, a coverage of the prostate cancer question texts in the corpus dataset is determined based on the user intention of each prostate cancer question text, and each prostate cancer question text in the corpus dataset is classified based on the user intention of each prostate cancer question text, so as to obtain a category attribute corresponding to each prostate cancer question text. After determining the user intention of each question text, the coverage of questions that can be covered by the corpus dataset can be determined based on all user intentions, and the corresponding category attribute of each question text can be determined, the category attributes refer to the lifestyle related to prostate cancer, such as smoking, drinking, staying up late, etc. The question-answering knowledge base is constructed based on the coverage of the prostate cancer question texts in the corpus dataset and the corresponding category attribute of each prostate cancer question text. The question-answering knowledge base can include a coarse classifier and at least one fine classifier, the coarse classifier is used to determine the coverage of question texts of the question-answering knowledge base, the at least one fine classifier is used to determine the category attribute corresponding to each prostate cancer question text, and the question-answering knowledge base is used to feedback on user initiated interaction messages.

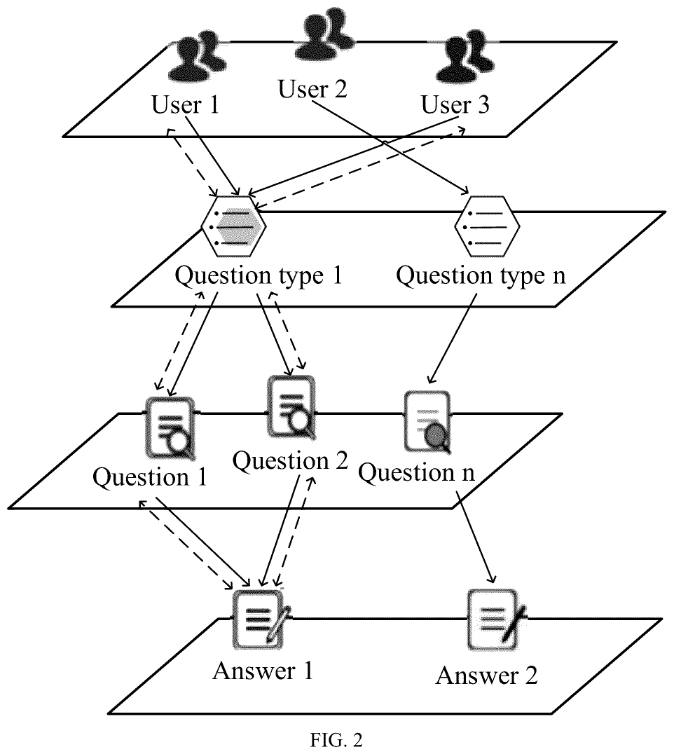

Exemplarily, as shown in FIG. 2, it is an interactive process between one or more ordinary users and the intelligent question-answering system for prostate cancer. When using the intelligent question-answering system for prostate cancer, first the type of question you want to know is inputted, such as “Whether drinking more than 100 mL of alcohol has an impact on prostate cancer”, the intelligent question-answering system for prostate cancer performs semantic parsing about the user input instruction, to extract the lifestyle of the keyword “drinking” and input it into the lifestyle knowledge base and the lifestyle knowledge graph to identify multiple questions 1, question 2, . . . question n which related to drinking problem, and using “drinking more than 100 mL” to determine question 1, then, based on the correlation between the question and the answer, the answer corresponding to determined question 1 can be acquired, and all the relevant data in the lifestyle knowledge base and the lifestyle knowledge graph regarding the question of “whether drinking more than 100 mL of alcohol has an impact on prostate cancer” can be acquired.

### Embodiment 2

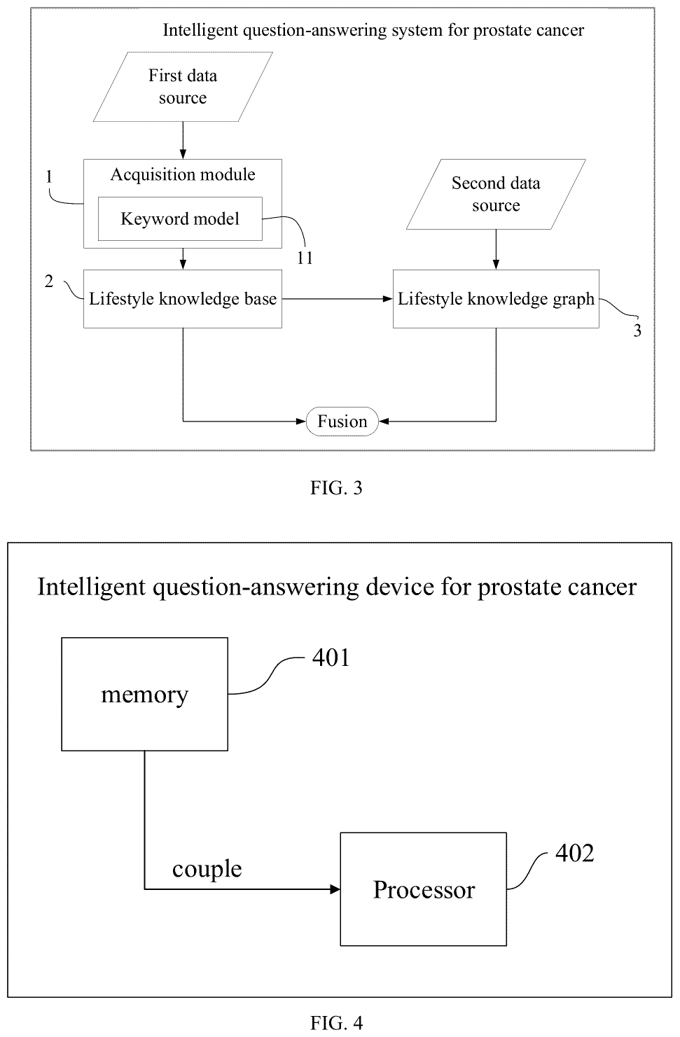

Please refer to FIG. 3, FIG. 3 is a block diagram of an intelligent question-answering system for prostate cancer according to an embodiment of the disclosure. The system includes an acquisition module 1, a lifestyle knowledge base 2 and a lifestyle knowledge graph 3.

The acquisition module 1 is configured to acquire lifestyle data for prostate cancer based on a preset first data source. The lifestyle knowledge base 2 is constructed by taking the lifestyle data for prostate cancer as metadata. The lifestyle knowledge graph 3 is constructed based on a preset second data source and the lifestyle knowledge base. The intelligent question-answering system for prostate cancer is implemented by fusing the lifestyle knowledge base and the lifestyle knowledge graph.

Specifically, the acquisition module 1 includes a keyword model 11 trained to be related to the lifestyle data for prostate cancer; the acquisition module 1 is configured to modularize data of the first data source to obtain multiple datasets, and extract the lifestyle data for prostate cancer from the multiple datasets by using the keyword model. Specifically, the first data source is implemented by objective global academic literature systems and websites. Exemplarily, the first data source of the embodiment can refer to the National Center for Biotechnology Information (NCBI) literature database and the Chinese National Knowledge Infrastructure (CNKI) literature database. After that, data from the above database are invoked through a data source interface, due to the inventor found that the impact of lifestyle on prostate cancer is of utmost importance, in these databases, the lifestyle data for prostate cancer are mainly filtered, the lifestyle data for prostate cancer can refer to the lifestyle data affected the probability and clinical outcomes of prostate cancer, the lifestyle data not only include names, types, subclasses, doses and exposure time of the lifestyle and disease classification and impact relationship of prostate cancer, but also include background information such as the population, race, quantity, data sources, literature names, publication dates, years, and journals involved in the research. Exemplarily, the lifestyle data include personal background characteristics (i.e., family history, etc.), behavioral habits (i.e., smoking, alcohol, etc.), environmental factors (i.e., bisphenol A, insecticides, etc.), minerals (i.e., selenium, calcium, etc.), vitamins (i.e., serum retinol (β-Carotenoids, etc.), drugs (i.e., antidiabetic, statins, etc.), diseases (i.e., diabetes, metabolic syndrome, etc.), social factors (i.e., long-term work, stress, etc.), food (i.e., carbohydrate, fat intake, etc.), physiological and biochemical factors (i.e., endocrine, hormone), etc. The data are manually retrieved, collected and extracted, cleaned, filtered, and structured, the content of data collection not only include names, types, subclasses, doses and exposure times of lifestyle, as well as disease classification and impact relationships of prostate cancer, but also include background information such as the population, race, quantity, data sources, literature names, publication dates, years, and journals involved in the research.

The acquiring lifestyle data for prostate cancer is implemented by: training a keyword model related to the lifestyle data for prostate cancer based on a preset machine learning training method, the keyword model includes multiple keywords and keyword-related statements of the above lifestyle data. The training model can be continuously updated and upgraded based on the update from the first data source, and then, the data from a calling interface of the first data source is modularized to obtain multiple datasets. Modularization is the most important feature of metadata, the key of the modularization is to divide resource objects into several entities according to the actual use needs. The description of resource refers to combination and description of multiple different entities, that is, the multiple datasets of different source types can be obtained, and the lifestyle data for prostate cancer can be extracted from the multiple datasets by using the trained keyword model, the extracted method can include inputting the datasets into the training model, performing automatically outputting on the matched lifestyle data for prostate cancer based on history training results.

In an embodiment, the lifestyle knowledge base 2 is implemented by: standardizing the lifestyle data for prostate cancer to obtain standard data, and structuring the standard data to obtain the lifestyle knowledge base with index relationships. In order to achieve user end conversion of the acquired lifestyle data for prostate cancer, the lifestyle data for prostate cancer is standardized to obtain standard data, the standardized method can be implemented by manually searching, collecting and extracting, cleaning, and filtering to uniformly define identifiers, data types, data lengths, and other information for different sources of data. After that, the standard data is structured to obtain the lifestyle knowledge base with index relationships, the structuration is primarily aimed at the user, the standard data can be transformed into recognizable and understandable data for the system, exemplarily, standardized texts of the metadata is transformed to XML Schema formal description files, various resource metadata is transformed and encapsulated to XML files based on XML Schema, so as to support automatic identification, understanding and verification of the XML files by computers. On the basis of the above data preparation, scientific relationships between various lifestyles and prostate cancer are sorted out manually to form an index relationship list, so as to form the lifestyle knowledge base.

In an embodiment, the lifestyle knowledge graph 3 is implemented by: filtering data of the second data source to obtain prostate science popularization data related to lifestyle data for prostate cancer by using the keyword model, and allocating the prostate science popularization data to obtain the lifestyle knowledge graph according to a preset graph rule. In order to strengthen the objectivity of data, the embodiment uses the second data source as an access method of data source, further concretizing the lifestyle knowledge base. The second data source can be derived from authoritative and widely global science popularization platforms such as Wikipedia. The data are acquired through the second data source to increase the data diversity and authority of the lifestyle knowledge base. Specifically, the data can include basic information about each lifestyle acquired from the second data source, the basic information includes synonyms, sources, basic descriptions, and other information about lifestyle, the data can further include the description, staging, Gleason score, and other information of prostate cancer in the clinical guidelines of prostate cancer. The acquired method can include filtering data of the second data source to obtain prostate science popularization data related to the lifestyle data for prostate cancer by using the keyword model, and allocating the prostate science popularization data to obtain the lifestyle knowledge graph according to a preset graph rule, the graph rule can include an inference rule, the graph rule can include using description logic for description. The description logic is a formal knowledge expression method based on logic. The description logic defines concepts, relationships, entities and a series of operators used to describe and constrain entity relationships, so as to establish a relationship between the acquired data and prostate cancer, and construct the lifestyle knowledge graph by using the relationship.

The intelligent question-answering system for prostate cancer is implemented by fusing the lifestyle knowledge base and the lifestyle knowledge graph, which specifically implemented by the following contents. The prostate science popularization data in the lifestyle knowledge graph and the lifestyle data for prostate cancer in the lifestyle knowledge base are taken as a corpus dataset, the corpus dataset at least includes prostate cancer question texts and prostate cancer answer texts, semantic parsing is performed on each prostate cancer question text in the corpus dataset to obtain a user intention of each prostate cancer question text, the semantic parsing can be implemented through alphabetic string parsing, a coverage of the prostate cancer question texts in the corpus dataset is determined based on the user intention of each prostate cancer question text, and each prostate cancer question text in the corpus dataset is classified based on the user intention of each prostate cancer question text, so as to obtain a category attribute corresponding to each prostate cancer question text. After determining the user intention of each question text, the coverage of questions that can be covered by the corpus dataset can be determined based on all user intentions, and the corresponding category attribute of each question text can be determined, the category attributes refer to the lifestyle related to prostate cancer, such as smoking, drinking, staying up late, etc. The question-answering knowledge base is constructed based on the coverage of the prostate cancer question texts in the corpus dataset and the corresponding category attribute of each prostate cancer question text. The question-answering knowledge base can include a coarse classifier and at least one fine classifier, the coarse classifier is used to determine the coverage of question texts of the question-answering knowledge base, the at least one subdivision classifier is used to determine the category attribute corresponding to each prostate cancer question text, and the question-answering knowledge base is used to feedback on user initiated interaction messages.

Exemplarily, the intelligent question-answering system for prostate cancer can use an iframe structure on a main page, the main page is divided into three parts, on the left is a prostate cancer problem classification navigation, on the right is a lifestyle overview list, and on the top is a search form. The classification navigation and the overview list program are relatively simple, so omitting them, the search form dialog box can add search prompts. The form program is as follows: <form method=“POST” action=“faq-list. asp” target=“main”><p>FAQ Search<input type=“text” name=“KeyWord” size=” 30 “value=” Please input the search term, terms are separated by spaces “on focus=” if (value=‘Please enter the search term, terms are separated by spaces’){value=“}” onblur=“if (value==”) {value=‘Please enter the search term, terms are separated by spaces’ }” style=” color: #808080; font-size: 10 pt “><input type=”submit” value=“Search” name=′B1″></p></form>.

### Embodiment 3

Please refer to FIG. 4, FIG. 4 is a schematic structural diagram of an intelligent question-answering device for prostate cancer according to an embodiment of the disclosure. Specifically, the intelligent question-answering system for prostate cancer described in FIG. 4 can be applied to systems such as chat robot terminals, mobile device terminals, portable mobile device terminals, personal wearable device terminals, or web page terminals. The application system of the intelligent question-answering device for prostate cancer is not limited by the embodiments of the disclosure. As shown in FIG. 4, the device can include a memory 401 and a processor 402.

The memory 401 stores executable program codes.

The processor 402 is coupled to the memory 401.

The processor 402 invokes the executable program codes in the memory 401 to execute the implementation method of the intelligent question-answering system for prostate cancer described in embodiment 1.

### Embodiment 4

The embodiment of the disclosure discloses a computer-readable storage medium, the computer-readable storage medium stores computer programs that are used for electronic data interchange, and the computer programs make a computer execute the implementation method of the intelligent question-answering system for prostate cancer described in embodiment 1.

### Embodiment 5

The embodiment of the disclosure discloses a computer program product, the computer program product includes a non-transitory computer-readable storage medium that stores computer programs, and the computer programs make a computer execute the implementation method of the intelligent question-answering system for prostate cancer described in embodiment 1.

The above-described embodiments are only illustrative, the modules described as separate components may or may not be physically separate, the components displayed as modules may or may not be physical modules, and they can be located in one place or distributed across multiple network modules. Some or all modules can be selected according to actual needs to achieve the purpose of the embodiment. those skilled in the art can understand and implement without creative labor.

Based on the specific description of the above embodiments, those skilled in the art can clearly understand that various embodiments can be implemented through software and necessary universal hardware platforms, it can also be achieved through hardware certainly. Based on this understanding, the above technical solutions, or the parts that contribute to relating art, can be reflected in the form of software products, the computer software products can be stored in the computer-readable storage medium, and the storage medium includes read-only memory (ROM), random access memory (RAM), programmable read-only memory (PROM), erasable programmable read-only memory (EPROM), one-time programmable read-only memory (OTPROM), electrically-erasable programmable read-only memory (EEPROM), compact-disc read-only memory (CD-ROM) and other optical disk memory, disk memory, magnetic tape memory, or any other computer-readable medium that can be used to carry or store data.

Finally, it should be noted that: the disclosure disclosed the intelligent question-answering system for prostate cancer and the implementation method thereof are only the preferred embodiments of, it is only used to illustrate the technical solution of the present invention, not to limit it; although the disclosure has been described in detail concerning the aforementioned embodiments, those skilled in the art should understand; they can still modify the technical solutions recorded in the aforementioned embodiments, or equivalently replace some of the technical features; and these modifications or replacements do not separate the essence of the corresponding technical solutions from the spirit and scope of the various embodiments of the disclosure.

What is claimed is:

1. An implementation method of an intelligent question-answering system for prostate cancer, comprising:
acquiring lifestyle data for prostate cancer based on a first data source;
constructing a lifestyle knowledge base by taking the lifestyle data for prostate cancer as metadata;
constructing a lifestyle knowledge graph based on a second data source and the lifestyle knowledge base; and
fusing the lifestyle knowledge base and the lifestyle knowledge graph to obtain the intelligent question-answering system for prostate cancer.

2. The implementation method of the intelligent question-answering system for prostate cancer according to claim 1, wherein acquiring the lifestyle data for prostate cancer based on the first data source, comprises:
training a keyword model related to the lifestyle data for prostate cancer;
modularizing data of the first data source to obtain a plurality of datasets; and
extracting the lifestyle data for prostate cancer from the plurality of datasets by using the keyword model.

3. The implementation method of the intelligent question-answering system for prostate cancer according to claim 2, wherein constructing the lifestyle knowledge base by taking the lifestyle data for prostate cancer as the metadata, comprises:
standardizing the lifestyle data for prostate cancer to obtain standard data; and
structuring the standard data to obtain the lifestyle knowledge base with index relationships.

4. The implementation method of the intelligent question-answering system for prostate cancer according to claim 3, wherein constructing the lifestyle knowledge graph based on the second data source and the lifestyle knowledge base, comprises:
filtering data of the second data source to obtain prostate science popularization data related to the lifestyle data for prostate cancer by using the keyword model; and
allocating the prostate science popularization data to obtain the lifestyle knowledge graph according to a graph rule.

5. The implementation method of the intelligent question-answering system for prostate cancer according to claim 4, wherein fusing the lifestyle knowledge base and the lifestyle knowledge graph to obtain the intelligent question-answering system for prostate cancer, comprises:
taking the prostate science popularization data in the lifestyle knowledge graph and the lifestyle data for prostate cancer in the lifestyle knowledge base as a corpus dataset, wherein the corpus dataset at least comprises prostate cancer question texts and prostate cancer answer texts;
performing semantic parsing on each of the prostate cancer question texts in the corpus dataset to obtain a user intention of each prostate cancer question text, determining a coverage of the prostate cancer question texts in the corpus dataset based on the user intention of each prostate cancer question text, and classifying each prostate cancer question text in the corpus dataset based on the user intention of each prostate cancer question text to obtain a category attribute corresponding to each prostate cancer question text; and
constructing a question-answering knowledge base based on the coverage of the prostate cancer question texts in the corpus dataset and the corresponding category attribute of each prostate cancer question text.

6. An intelligent question-answering system for prostate cancer, comprising:
an acquisition module, configured to acquire lifestyle data for prostate cancer based on a first data source;
a lifestyle knowledge base, constructed by taking the lifestyle data for prostate cancer as metadata; and
a lifestyle knowledge graph, constructed based on a second data source and the lifestyle knowledge base; and
wherein the intelligent question-answering system for prostate cancer is implemented by fusing the lifestyle knowledge base and the lifestyle knowledge graph.

7. The intelligent question-answering system for prostate cancer according to claim 6, wherein the acquisition module comprises: a keyword model trained to be related to the lifestyle data for prostate cancer; and
wherein the acquisition module is configured to modularize data of the first data source to obtain a plurality of datasets, and extract the lifestyle data for prostate cancer from the plurality of datasets by using the keyword model.

8. The intelligent question-answering system for prostate cancer according to claim 7, wherein the lifestyle knowledge base is implemented by:
standardizing the lifestyle data for prostate cancer to obtain standard data; and
structuring the standard data to obtain the lifestyle knowledge base with index relationships.

9. The intelligent question-answering system for prostate cancer according to claim 8, wherein the lifestyle knowledge graph is implemented by:
filtering data of the second data source to obtain prostate science popularization data related to lifestyle data for prostate cancer by using the keyword model; and
allocating the prostate science popularization data to obtain the lifestyle knowledge graph according to a graph rule.

10. The intelligent question-answering system for prostate cancer according to claim 9, wherein the intelligent question-answering system for prostate cancer is implemented by fusing the lifestyle knowledge base and the lifestyle knowledge graph, comprises:
taking the prostate science popularization data in the lifestyle knowledge graph and the lifestyle data for prostate cancer in the lifestyle knowledge base as a corpus dataset, wherein the corpus dataset at least comprises prostate cancer question texts and prostate cancer answer texts;
performing semantic parsing on each of the prostate cancer question texts in the corpus dataset to obtain a user intention of each prostate cancer question text, determining a coverage of the prostate cancer question texts in the corpus dataset based on the user intention of each prostate cancer question text, and classifying each prostate cancer question text in the corpus dataset based on the user intention of each prostate cancer question text to obtain a category attribute corresponding to each prostate cancer question text; and
constructing the question-answering knowledge base based on the coverage of the prostate cancer question texts in the corpus dataset and the corresponding category attribute of each prostate cancer question text.
